(19) United States
(12) Patent Application Publication
(10) Pub. No.: US 2007/0244031 A1
(43) Pub. Date: Oct. 18, 2007

(54) Methods and Compositions for Homozygous Gene Inactivation Using Collections of Pre-Defined Nucleotide Sequences Complementary Chromosomal Transcripts

(76) Inventors: Quan Lu, Mountain View, CA (US); Stanley N. Cohen, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/582,050

(22) PCT Filed: Jan. 25, 2005
(86) PCT No.: PCT/US05/02379

§ 371 Date: Apr. 12, 2007

Related U.S. Application Data
(60) Provisional application No. 60/539,908, filed on Jan. 27, 2004.

(51) Int. Cl. A61K 31/00; C07H 21/00; C12N 15/00; C12N 5/00; C12Q 1/68

(52) U.S. Cl. 514/1; 435/254.1; 435/320.1; 435/325; 435/375; 435/410; 435/6; 536/23.1

(57) ABSTRACT

Methods and compositions for performing homozygous gene inactivation assays are provided. A feature of the subject methods is the use of a library of constructs that synthesize predefined nucleic acids, where each constituent predefined nucleic acid of the library is of known sequence that corresponds to a sequence of a chromosomal transcript, e.g., where a representative embodiment of a predefined nucleic acid is an expressed sequence tag (i.e., EST). In certain embodiments, the subject libraries are produced using an amplification protocol that preserves the sequence representation profile of the template nucleic acids. The subject methods and compositions find use in a variety of different applications, including the identification of novel diagnostic and therapeutic genetic targets.

### CROSS-REFERENCE TO RELATED APPLICATIONS

Pursuant to 35 U.S.C. §119 (e), this application claims priority to the filing date of the U.S. Provisional Patent Application Ser. No. 60/539,908 filed Jan. 27, 2004; the disclosure of which is herein incorporated by reference.

### GOVERNMENT RIGHTS

This invention was made with Government support under contract N655236-99-1-5425 awarded by the Defense Advanced Research Projects Agency. The United States Government has certain rights in this invention.

### INTRODUCTION

### BACKGROUND OF THE INVENTION

In view of the rapidity of gene discovery that has resulted in the identification and sequencing of a large number of genes, determining the biological functions of genes is a major challenge in biotechnology today. To meet this challenge, a variety of different protocols have been developed to assign functions to previously identified genes and to identify genes that have biological functions of interest.

In organisms that contain a single copy of each gene, it is practical to identify genes that have particular functions by randomly inactivating genes using one of a variety of mutational methods, and then selecting or screening for individuals that acquire altered biological properties as a result of gene inactivation. However, higher organisms contain two copies of each gene, and mutation of only one copy usually does not result in altered biological properties, as the remaining copy continues to function. Inactivation of both copies of the same gene in a single cell using mutational methods generally is impractical unless the sequence of the gene has been previously determined, since the frequency of random mutagenesis by standard approaches is too low for the same cell to acquire mutations in both gene copies.

This problem has given rise to the field of genomics, in which coding sequences of genes commonly are first cloned and sequenced and the sequences obtained are then used to find or infer function. However, there remains an important need for direct approaches to the identification of mammalian cell genes having particular functions. Specifically, the use of high-throughput cellular assays for altered gene function plus methods for discovery of gene function by concurrent alteration of the activity of both copies of genes in mammalian cells are desired.

Several methods have been used or proposed for the inactivation of both copies of mammalian cell genes. In such methods, cells which acquire a phenotype of interest that results from gene inactivation are isolated, and knowledge of gene function is derived from the phenotype observed when the gene is inactivated. In certain applications, the gene of interest whose function is to be assayed is of known sequence, while in other applications, part or all of the sequence of the gene is unknown. In the latter case, acquisition of a cell phenotype as a consequence of inactivation is used to both discover the gene and identify its function. When the sequence of a gene is previously known, a variety of approaches for gene inactivation are available, which approaches are inapplicable in the inactivation of genes of unknown sequence, as reviewed in greater detail below.

As noted above, methods for identifying mammalian genes that have particular functions of interest by gene inactivation suffer from several drawbacks. First of all, as noted above, mammalian cells are diploid for most genes, and the identification of cells containing lesions that produce recessive phenotypes normally requires that both cellular alleles of the gene be inactivated. Commonly, the inactivation step results in inactivation of only a single gene copy. The other gene copy may still be expressed, and the phenotype of the cell containing the inactivated gene may be indistinguishable from the wild-type phenotype. While homozygous inactivation of previously cloned genes has been accomplished by gene targeting and homologous recombination combined with appropriate selection techniques, this approach normally cannot be taken unless the gene has been cloned previously or its sequence known. Similarly, homozygous inactivation of multiple alleles of genes can be accomplished using synthesized RNA or DNA oligonucleotides complementary to part or all of the sequence of the particular gene to be inactivated, but again, this approach requires prior cloning of the gene and/or knowledge of its sequence.

For genes that have not yet been cloned, gene expression in antisense orientation can also be an effective way to suppress the activity and thus the function of a target gene. Previous approaches have exploited such antisense gene expression as a genetic screening tool. In certain of these studies, populations of cells that contain cDNAs expressed in antisense orientation were generated and then were screened for a phenotype of interest. One problem with this antisense cDNA approach is that genes are not equally represented in the pool of cDNAs (e.g., some genes such as actin are much more abundant than other genes such as rare enzymes). Even in a so-called “normalized” cDNA pool this problem still exists, although to a lesser extent. The unequal representation of genes in cDNA libraries seriously undermines the applicability and efficiency of library screening since it dramatically increases the number of clones needed to achieve complete coverage of the genes in the genome. In a practical sense, genes expressed to a relatively small extent may not be represented in cDNA libraries of attainable size.

One approach for random inactivation of genes in mammalian cells involves the use of viral vectors to introduce into, and insert chromosomally in, mammalian cells promoters that initiate transcription into the chromosomal DNA sequence that flanks the site of insertion of the vector. While this approach has proved to be successful in the identification of genes having phenotypes of interest, actual isolation and validation of the function of the cognate gene can be cumbersome.

There is thus a continued need for the development of mammalian cell-based gene inactivation methods where the above disadvantages are overcome. The present invention satisfies this need.

Relevant Literature

U.S. Pat. Nos. 5,679,523; 6,376,241 and 6,413,776; as well as published PCT Application Nos. WO 02/070684; WO 02/092807 and WO 02/092808. See also Gudkov et al., Proc. Nat'l Acad. Sci USA (1994) 91:3744-3748; Kimchi, Methods Mol. Biol. (2003) 222: 399-412; Li & Cohen, Cell (1996). 85: 319-329; Pierce & Ruffner, Nuc. Acids Res. (1998)26:5093-5101; Berns et al., Nature (2004) 428:431-437 and Paddison et al., Nature (2004) 428: 427-431.

### SUMMARY OF THE INVENTION

Methods and compositions for performing homozygous gene inactivation assays are provided. A feature of the subject methods is the use of a library of constructs that synthesize predefined nucleic acids, where each constituent predefined nucleic acid of the library is of known sequence that corresponds to a sequence of a chromosomal transcript, e.g., where a representative embodiment of a predefined nucleic acid is an expressed sequence tag (i.e., EST). In certain embodiments, the subject libraries are produced using an amplification protocol that preserves the sequence representation profile of the template nucleic acids. The subject methods and compositions find use in a variety of different applications, including the discovery and identification of novel diagnostic and therapeutic genetic targets.

### BRIEF DESCRIPTION OF THE FIGURES

FIG. 1(a) provides a schematic diagram of the lentiviral expression vector pLEST, employed in a representative embodiment of the subject invention. FIG. 1(b) provides a schematic diagram of a procedure for construction of pLEST-based EST libraries according to an embodiment of the subject invention. FIG. 1(c) provides a general scheme for an EST library-screening process according to an embodiment of the subject invention.

### DESCRIPTION OF THE SPECIFIC EMBODIMENTS

Methods and compositions for performing homozygous gene inactivation assays are provided. A feature of the subject methods is the use of a library of constructs that express predefined nucleic acids, where each constituent predefined nucleic acid of the library is of known sequence that corresponds to a sequence or sequences of a chromosomal transcript, e.g., where a representative embodiment of a predefined nucleic acid is an expressed sequence tag (i.e., EST). In certain embodiments, the subject libraries are produced using an amplification protocol that preserves the sequence representation profile of the template nucleic acids from which the library is produced. The subject methods and compositions find use in a variety of different applications, including functional genomic applications, e.g., for the discovery and identification novel diagnostic and therapeutic genetic targets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, an overview of the invention will first be provided. Next, a more in-depth discussion of representative methods of producing the subject libraries is provided, followed by further elaboration of the libraries produced using the subject representative methods, as well as representative applications in which the subject libraries find use, is provided.

Overview

As summarized above, the subject invention provides methods and compositions for use in homozygous gene inactivation assays. A feature of the subject methods is the use of cells containing a library of predefined nucleic acids. The library of predefined nucleic acids is a pooled or combined collection of nucleic acids, where each constituent nucleic acid member of the library is of known sequence and corresponds to a known chromosomal transcript. Individual constituents may, as desired, be present in equal or unequal amounts. A further feature of the subject libraries is that each constituent member nucleic acid of the library is present in a vector, such as the vectors described below. Furthermore, each constituent member of the library is not immobilized on the surface of the solid support, such that the library is distinguished from arrays of immobilized nucleic acid, including fluid arrays. In many embodiments, the libraries of the subject invention are predefined pooled collections of distinct nucleic acid vectors, wherein each constituent member of the pooled collection includes an expression cassette that corresponds to a chromosomal transcript of known sequence. In certain embodiments, each constituent member is present in a known relative amount to all other constituent members of said pooled collection.

The total number of distinct or different nucleic acid members of the library may vary, but in certain embodiments is at least about 100, such as at least about 500, including at least about 1000 or more, and in certain embodiments may be as high as 5,000; 10,000; 50,000; or more. While the length of the predefined nucleic acid members of the libraries may vary, in certain embodiments the length is at least about 20 nt, such as at least about 100 nt, including at least about 200 nt.

As mentioned above, a feature of the libraries is that they consist of pooled collections of predefined nucleic acids. As such, each constituent predefined nucleic acid member of the libraries is of known sequence and corresponds to a known chromosomal transcript. By “known sequence” is meant the nucleotide sequence of the predefined nucleic acid is already determined. In other words, the predefined nucleic acid is a pre-sequenced nucleic acid. By “corresponds to a known chromosomal transcript” is meant that the predefined nucleic acid may be expressed, e.g., from a suitable vector, as a nucleic acid that includes a sequence found in the complement nucleic acid of a known chromosomal transcript, i.e, at least a segment of a chromosomal transcript. As such, the term corresponds to includes situations where the vector, e.g., expression cassette thereof, includes at least a segment of a chromosomal transcript of known sequence, where in certain embodiments the whole chromosomal segment may be present in the vector. In certain embodiments, the predefined nucleic acid may be transcribed into a RNA product that includes a sequence found in the complement of a known mRNA molecule (which is known to exist but may or may not be fully sequenced An example of such an embodiment is a library of ESTs, as described more fully below, where the predefined nucleic acid is an EST that is present in a vector and is transcribed into an RNA molecule that is the complement of at least a portion of the mRNA molecule from which the EST was derived, and as such includes a sequence found in the complement of a known (but not sequenced) chromosomal transcript (i.e., mRNA).

In certain embodiments, the constituent members of the libraries are also present in known amounts. More specifically, each member of the library is present in a known relative amount to the other members of the library, i.e., where the relative amount of a given member is known with respect to the other members of the library. In certain representative embodiments, the constituent members of the library are present in equal amounts, whereas in other embodiments the amounts may by choice or circumstance be unequal. In certain embodiments, the absolute or quantitative amount of each member of the library is known.

The libraries of the subject invention find use in homozygous gene inactivation assays, including random homozygous gene inactivation assays, as further described below. Briefly, in such methods a library according to the present invention is contacted with a cellular population under appropriate conditions such that each member of the library is introduced into a member of the cellular population. Those members of the library that are introduced into a cell which contains the chromosomal transcript target of their predefined nucleic acid then modulate, e.g., at least reduce if not completely inhibit or inactivate, functioning of the chromosomal region from which the target transcript arises. The resultant phenotype of such cells can then be evaluated to determine gene function of the target chromosomal transcript. Such methods are described in further detail below in connection of the representative EST library embodiments of the subject invention.

Following the above general overview of the invention, the invention will now be more fully described in terms of a representative embodiment for preparing the subject libraries, libraries produced by these representative methods, and representative applications in which these libraries may find use.

A Representative Method of Library Production

In the following representative library production method, the subject invention provides methods for producing nucleic acid libraries, e.g., EST libraries, from an initial set of separate nucleic acids. As reviewed in more detail below, the constituent nucleic acid members of the libraries produced using the subject methods are generally deoxyribonucleic acids (DNA). In these representative embodiments, the initial set of separate nucleic acids used to produce the subject libraries is a set of expressed sequences tags (ESTs), where the sequences of the constituent expressed sequence tag members of the initial set are found in the produced non-cellular nucleic acid library, such that the produced non-cellular nucleic acid library is a non-cellular EST library. By non-cellular nucleic acid library is meant a collection or plurality of nucleic acids of different sequence, i.e., a collection or set of distinct nucleic acids, that is not present inside of a cell, i.e., is present in an environment that is cell-free. Because the library in this representative embodiment is an EST library, all of the members of the library are of known sequence and correspond to a known chromosomal transcript, such that all of the members are predefined.

In practicing the subject methods of this representative embodiment, the first step is to divide an initial set of separate nucleic acids into two or more pooled collections of nucleic acids of limited size. The initial set of separate nucleic acids is an initial set of distinct nucleic acids of differing sequence, where any two given nucleic acid members in a given set are considered distinct or different if they comprise a stretch of at least 50, usually at least 100, nucleotides in length in which the sequence similarity is less then 95% or lower, as determined using the FASTA program (default settings). By “separate” is meant that all the members of the initial set are isolated from one another, such that they are not physically combined into a single composition. For example, each member of the initial set may be present in its own physical containment means, e.g., tube, well, etc.

Typically, each member of the initial set is present in a nucleic acid composition that includes the member present in a vector nucleic acid. A variety of different nucleic acid vectors are known, where representative vectors include, but are not limited to: plasmids, viral vectors, and the like. Where convenient, the vector for each member nucleic acid may be present in a cell, e.g., a bacterial cell, as is known in the art. When the nucleic acid member is present in a cell, the nucleic acid component is typically separated from the remainder of the cell, where any convenient protocol may be employed, including one or the numerous known nucleic acid extraction protocols employed in the art for separating nucleic acids from other cellular constituents.

The number of distinct nucleic acids in the initial set may vary widely, but is typically at least about 100 or more, such as at least about 1000 or more, including at least about 5000 or more. In many embodiments, the number of distinct nucleic acids in the sets ranges from about 100 to about 100,000, including from about 10,000 to about 100,000, such as from about 30,000 to about 60,000.

The initial set of separate distinct nucleic acids is divided into two or more collections or pools, e.g., fractions, of nucleic acids. In other words, two or more different collections of nucleic acids are produced from the initial set of nucleic acids, where the collections, pools or fractions produced in this step of the subject methods are physical mixtures of the distinct nucleic acids, such that the distinct nucleic acids of a given collection produced in this step are present in a single composition that is a combination of the nucleic acids, i.e., the nucleic acids of a given pool or collection are not physically separated from each other. The total number of distinct nucleic acids present in a given pool produced in this step of the subject methods may vary, but typically does not exceed about 200, where the number may not exceed about 150, and in certain embodiments does not exceed about 100.

The pools or collections are typically produced in this step by combining an appropriate number of distinct nucleic acids from the initial set of separate nucleic acids. Typically, known amounts of each distinct nucleic acid of the initial set are combined in this step of the subject methods, where the amounts typically range from about 1 ng to about 1000 ng, such as from about 10 ng to about 50 ng, so that the total amount of nucleic acids in the produced pool or collection ranges from about 10 ng to about 10 ug, such as from about 1 ug to about 5 ug. The copy number of each distinct nucleic acid in the produced pool or collection may vary, but in many embodiments ranges from about 10⁹ to about 10¹², such as from about 10¹⁰ to about 10¹¹.

As mentioned above, the initial set is divided into two or more pools or collections of nucleic acids, as described above. The number of different pools or collections produced in this step necessarily varies, depending on the size of the initial set and the number of distinct sequences desired in each pool or collection. In many embodiments, the number of pools or collections produced in this step ranges from about 5 to about 1,000, such as from about 100 to about 500.

Regardless of the total number of different pools or collections produced in this step, one characteristic or feature of each member of the total number of different pools, as well as the sum set of all of the different pools, is the sequence representation profile of the each pool and the sum set of all of the pools. By sequence representation profile is meant the amount, e.g., relative and/or quantitative, of each distinct nucleic acid in the pool or sum set of pools. The sequence representation profile may also be viewed as the complexity of the pool or summed set of pooled nucleic acids. In certain embodiments, each of the distinct nucleic acids may be present in substantially equal, if not equal, amounts, such that the pool or sum set including the same has a sequence representation profile that is “equimolar” with respect to its constituent members. By equal amounts is meant that the amounts of any two given distinct nucleic acids in the pool/sum set of pools do not vary by more than about 5-fold, typically by not more than about 3-fold, e.g., by not more than about 1-fold. In yet other embodiments, the amounts of any two given nucleic acids may not be at least substantially equal. Whether or not the amounts of the distinct nucleic acids in the pools and sum sets thereof are or are not equal, the pools and sum sets thereof may be characterized by having a sequence representation profile or complexity, as described above, i.e., an initial or first sequence representation profile or complexity.

Following the above-described first step of producing pools or collections of nucleic acids from the initial source, each pool or collection is then amplified to produce an amplified pool or collection, such that an amplified pool or collection of nucleic acids is produced from each initial pool or collection of nucleic acids. By amplified pool is meant a pool that has an increased copy number of a given nucleic acid as compared to the copy number of that nucleic acid in the initial pool from which the amplified pool is produced, where the magnitude of increase may vary depending on the amplification protocol employed, and is, in many embodiments, at least about 10-fold, such as at least about 100-fold, including at least about 1,000-fold.

A variety of amplification protocols are known in the art and may be employed, so long as the amplification maintains the sequence representation profile of the pool being amplified, and therefore the combined set of the pools or collections of nucleic acids. Amplification protocols of interest include both linear and geometric amplification protocols. A particular amplification protocol of interest is the polymerase chain reaction, and applications based thereon. The polymerase chain reaction (PCR), in which a nucleic acid primer extension product is enzymatically produced from template DNA,.is well known in the art, being described in U.S. Pat. Nos. 4,683,202; 4,683,195; 4,800,159; 4,965,188 and 5,512,462, the disclosures of which are herein incorporated by reference.

In this step of the subject methods, the pool or collection of nucleic acids, which serves as the template nucleic acid, is contacted with primer or primers, one or more nucleic acid polymerases, and other reagents, into a reaction mixture. The amount of template nucleic acid, i.e, pool or collection of nucleic acids, that is combined with the other reagents may range from about 1 molecule to 1 pmol, usually from about 50 molecules to 0.1 pmol, and more usually from about 0.01 amol to 100 f mol in certain representative embodiments.

The oligonucleotide primers with which the template nucleic acid (hereinafter referred to as template DNA for convenience) is contacted are of sufficient length to provide for hybridization to complementary template DNA under annealing conditions (described in greater detail below), and are of insufficient length to form stable hybrids with template DNA under polymerization conditions. The primers are generally at least about 10 nt in length, usually at least about 15 nt in length and more usually at least about 16 nt in length and may be as long as about 30 nt in length or longer, where the length of the primers generally ranges from about 18 nt to about 50 nt in length, such as from about 20 nt to about 35 nt in length.

As discussed above, the template DNA is contacted with a primer composition. The primer composition may vary. For example, where the distinct nucleic acid members of the pool or collection to be amplified are present in vectors that include at least one bounding or flanking universal priming site, the same primer may be employed to amplify each distinct constituent member of the pool. The template DNA may be contacted with a single primer or a set of two primers, depending on whether linear or exponential amplification of the template DNA is desired. Where a single primer is employed, the primer will typically be complementary to one of the 3′ ends of the template DNA and when two primers are employed, the primers will typically be complementary to the two 3′ ends of the double stranded template DNA. In those embodiments where a flanking or universal priming site is not present or available for use, a “gene-specific” primer collection made up of a primer or primer pair (as described above) for each distinct nucleic acid in the pool is employed (e.g., a collection of 100 different primers or primer pairs (depending on whether linear or geometric amplification is desired, respectively) is employed—one for each constituent member in the pool or collection).

The subject amplification methods of these PCR embodiments employ at least one Family A polymerase, and in many embodiments a combination of two or more different polymerases, usually two, different polymerases. The polymerases employed will typically, though not necessarily, be thermostable polymerases. The polymerase combination with which the template DNA and primer is contacted will comprise at least one Family A polymerase and, in many embodiments, a Family A polymerase and a Family B polymerase, where the terms “Family A” and “Family B” correspond to the classification scheme reported in Braithwaite & Ito, Nucleic Acids Res. (1993) 21:787-802. Family A polymerases of interest include: Thermus aquaticus polymerases, including the naturally occurring polymerase (Taq) and derivatives and homologues thereof, such as Klentaq (as described in Proc. Natl. Acad. Sci. USA (1994) 91:2216-2220); Thermus thermophilus polymerases, including the naturally occurring polymerase (Tth) and derivatives and homologues thereof, and the like. Family B polymerases of interest include Thermococcus litoralis DNA polymerase (Vent) as described in Perler et al., Proc. Natl. Acad. Sci. USA (1992) 89:5577; Pyrococcus species GB-D (Deep Vent); Pyrococcus furiosus DNA polymerase (Pfu) as described in Lundberg et al., Gene (1991) 108:1-6, Pyrococcus woesei (Pwo) and the like. Of the two types of polymerases employed, the Family A polymerase will be present in an amount greater than the Family B polymerase, where the difference in activity will usually be at least 10-fold, and more usually at least about 100-fold. Accordingly, the reaction mixture prepared upon contact of the template DNA, primer, polymerase and other necessary reagents, as described in greater detail below, will typically comprise from about 0.1 U/μl to 1 U/μl Family A polymerase, usually from about 0.2 to 0.5 U/μl Family A polymerase, while the amount of Family B polymerase will typically range from about 0.01 mU/μl to 10 mU/μl, usually from about 0.05 to 1 mU/μl and more usually from about 0.1 to 0.5 mU/μl, where “U” corresponds to incorporation of 10 nmol dNTP into acid-insoluble material in 30 min at 74° C.

Also present in the reaction mixture will be deoxyribonucleoside triphosphates (dNTPs). Usually the reaction mixture will comprise four different types of dNTPs corresponding to the four naturally occurring bases, i.e. dATP, dTTP, dCTP and dGTP. The reaction mixture will further comprise an aqueous buffer medium that may include one or more of: a source of monovalent ions, a source of divalent cations and a buffering agent. Any convenient source of monovalent ions, such as KCl, K-acetate, NH₄-acetate, K-glutamate, NH₄Cl, ammonium sulfate, and the like may be employed, where the amount of monovalent ion source present in the buffer will typically be present in an amount sufficient to provide for a conductivity in a range from about 500 to 20,000, usually from about 1000 to 10,000, and more usually from about 3,000 to 6,000 micromhos. The divalent cation may be magnesium, manganese, zinc and the like, where the cation will typically be magnesium. Any convenient source of magnesium cation may be employed, including MgCl₂, Mg-acetate, and the like. The amount of Mg²⁺ present in the buffer may range from 0.5 to 10 mM, but will preferably range from about 2 to 4 mM, more preferably from about 2.25 to 2.75 mM and will ideally be at about 2.45 mM. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, where most preferred is pH 7.3 at 72° C. Other agents which may be present in the buffer medium include chelating agents, such as EDTA, EGTA and the like.

In preparing the reaction mixture, the various constituent components may be combined in any convenient order. For example, the buffer may be combined with primer, polymerase and then template DNA, or all of the various constituent components may be combined at the same time to produce the reaction mixture.

Following preparation of the reaction mixture, the reaction mixture is subjected to a plurality of reaction cycles, where each reaction cycle comprises: (1) a denaturation step, (2) an annealing step, and (3) a polymerization step. The number of reaction cycles will vary depending on the application being performed, but will usually be at least 15, more usually at least 20 and may be as high as 60 or higher, where the number of different cycles will typically range from about 20 to 40. For methods where more than about 25, usually more than about 30 cycles are performed, it may be convenient or desirable to introduce additional polymerase into the reaction mixture such that conditions suitable for enzymatic primer extension are maintained.

The denaturation step comprises heating the reaction mixture to an elevated temperature and maintaining the mixture at the elevated temperature for a period of time sufficient for any double stranded or hybridized nucleic acid present in the reaction mixture to dissociate. For denaturation, the temperature of the reaction mixture will usually be raised to, and maintained at, a temperature ranging from about 85 to 100, usually from about 90 to 98 and more usually from about 93 to 96° C. for a period of time ranging from about 3 to 120 sec, usually from about 5 to 30 sec.

Following denaturation, the reaction mixture will be subjected to conditions sufficient for primer annealing to template DNA present in the mixture. The temperature to which the reaction mixture is lowered to achieve these conditions will usually be chosen to provide optimal efficiency and specificity, and will generally range from about 50 to 75, usually from about 55 to 70 and more usually from about 60 to 68° C. Annealing conditions will be maintained for a period of time ranging from about 15 sec to 30 min, usually from about 30 sec to 5 min.

Following annealing of primer to template DNA or during annealing of primer to template DNA, the reaction mixture will be subjected to conditions sufficient to provide for polymerization of nucleotides to the primer ends in manner such that the primer is extended in a 5′ to 3′ direction using the DNA to which it is hybridized as a template, i.e. conditions sufficient for enzymatic production of primer extension product. To achieve polymerization conditions, the temperature of the reaction mixture will typically be raised to or maintained at a temperature ranging from about 65 to 75, usually from about 67 to 73° C. and maintained for a period of time ranging from about 15 sec to 20 min, usually from about 30 sec to 5 min.

The above cycles of denaturation, annealing and polymerization may be performed using an automated device, typically known as a thermal cycler. Thermal cyclers that may be employed are described in U.S. Pat. Nos. 5,612,473; 5,602,756; 5,538,871; and 5,475,610, the disclosures of which are herein incorporated by reference.

In representative embodiments, the amplification protocol employed is one that employs maximal template and minimal cycles. By maximal template is meant that the amount of template employed in a given amplification reaction of 100 μl is at least about 0.1 ng, including at least about 1 ng, such as at least about 10 ng, and may range from about 0.1 ng to about lug, such as from about 10 ng to about 1 ug. By minimal cycles is meant less than about 25 cycles, such as less than about 20 cycles, where the number of cycles typically ranges from about 10 to about 30 cycles, such as from about 15 to about 18 cycles.

Following amplification of the two or more pools or collections of nucleic acids, the resultant amplified pools or collections are then combined into a single composition or mixture to produce the desired nucleic acid library. The amplified pools or collections may be combined using any convenient protocol, where the pools may be combined sequentially or simultaneously, as desired.

Representative Non-Cellular Nucleic Acid Libraries

As summarized above, the subject methods (as reviewed above) produce non-cellular nucleic acid libraries from an initial set of separate nucleic acids. The constituent nucleic acid members of the libraries produced using the subject methods are generally deoxyribonucleic acids (DNA). In many embodiments, the initial set of separate nucleic acids used to produce the subject libraries is a set of expressed sequences tags (ESTs), where the sequences of the constituent expressed sequence tag members of the initial set are found in the produced non-cellular nucleic acid library, such that the produced non-cellular nucleic acid library is a non-cellular EST library. By non-cellular nucleic acid library is meant a collection or plurality of nucleic acids of different sequence, i.e., a collection or set of distinct nucleic acids, that is not present inside of a cell, i.e., is present in an environment that is cell-free.

A feature of the nucleic acid libraries produced by the subject methods is that they have a sequence representation profile or complexity that is substantially the same as that of the initial pools/collections (as well as sum sets thereof) of nucleic acids, as described above. By “substantially the same as” is meant that the magnitude of any variation, if any, in an amount of any given nucleic acid in the final produced nucleic acid library as compared to the amount of the nucleic acid in the initial pool (and therefore combined set of pools) in which it is found does not exceed about 10-fold, and usually does not exceed about 2-fold.

In certain embodiments, the produced nucleic acids include a relatively large number of distinct nucleic acids in a relatively small amount of total nucleic acid. In such embodiments, the number of distinct nucleic acids in the library may be at least about 1,000, such as at least about 10,000, including at least about 100,000, in a total amount that does not exceed about 100 μg, such as an amount that does not exceed about 10 μg, including an amount that does not exceed about 1 μg. In certain of these embodiments, the ratio of the number of distinct nucleic acids in the library per amount of total nucleic acid in the library may range from about 10/μg to about 10,000/μg, such as from about 100/μg to about 1,000/μg, including from about 200/μg to about 500/μg.

In certain of these embodiments, despite the relatively small size of the libraries, the libraries are “genome-wide” libraries. In such embodiments, substantially all, if not all, of the sequences found in the parent organism genomic coding sequence from which the initial set of nucleic acids is obtained are present in the produced probe population. By substantially all is meant typically at least about 75%, such as at least about 80%, at least about 85%, at least about 90% or more, including at least about 95%, at least about 95% etc, of the total genomic coding sequence sequences of the parent organism are present in the produced library, where the above percentage values are number of bases in the produced library as compared to the totaf number of bases in the genomic source.

Such a library can be readily identified using a number of different protocols. One convenient protocol for determining whether a given library is a genome wide library is to screen the collection using a genome wide array of probe nucleic acids for the genomic source of interest. Thus, one can tell whether a given library is a genome wide library with respect to its genomic source by assaying the library with a genomic wide array for the genomic source. The genomic wide array of the genomic source is an array of probe nucleic acids in which substantially all of, if not all of, the mRNA transcripts encoded by the genomic source are represented, where by substantially all of is meant at least about 75%, such as at least about 80%, at least about 85%, at least about 90%, at least about 95% or higher. In such a genomic wide assay of a sample, a genome wide library is one in which substantially all of the array features on the array provide a positive signal, where by substantially all is meant at least about 50%, such as at least about 60, 70, 75, 80, 85, 90 or 95% (by number) or more.

The non-cellular nucleic acid libraries produced according to the subject methods and described above may be present in a number of different formats or configurations, i.e., constructs. Constructs are compositions that include a distinct nucleic acid sequence inserted into a vector, where such constructs may be used for a number of different applications, including propagation, screening, genome alteration, and the like, as described in greater detail below. Constructs made up of viral and non-viral vector sequences may be prepared and used, including plasmids, as desired. The choice of vector will depend on the particular application in which the nucleic acid is to be employed. Certain vectors are useful for amplifying and making large amounts of the desired DNA sequence. Other vectors are suitable for expression in cells in culture, e.g., for use in screening assays. Still other vectors are suitable for transfer and expression in cells in a whole animal, e.g., in the production of animal models of hyperproliferative diseases. The choice of appropriate vector is well within the ability of those of ordinary skill in the art. Of interest in certain embodiments are viral vectors. A variety of viral vector delivery vehicles are known to those of skill in the art and include, but are not limited to: adenovirus, herpesvirus, lentivirus, vaccinia virus and adeno-associated virus (AAV). Many such vectors are available commercially.

To prepare the constructs, the nucleic acid of interest is inserted into a vector, typically by means of DNA ligase attachment to a cleaved restriction enzyme site in the vector. Yet another means to insert the nucleic acids into appropriate vectors is to employ one of the increasingly employed recombinase based methods for transferring nucleic acids among vectors, e.g., the Creator™ system from Clontech; the Gateway™ system from Invitrogen, etc.

In certain embodiments, each distinct nucleic acid is present in a vector in the form of an expression cassette that includes the distinct nucleic acid. By expression cassette is meant a nucleic acid that includes a distinct nucleic acid sequence operably linked to a promoter sequence, where by operably linked is meant that expression of the coding sequence is under the control of the promoter sequence. In certain embodiments, the expression cassette is one that is transcribed into antisense RNA, such that the library is an antisense library. In these embodiments, the expression cassette is one in which the promoter sequences are oriented relative to the distinct nucleic acid sequence such that antisense RNA is transcribed from the expression cassette. In yet other embodiments, the expression cassette is one that is transcribed into sense RNA, such that the library is a sense library. In these embodiments, the expression cassette is one in which the promoter sequences are oriented relative to the distinct nucleic acid sequence such that sense RNA is transcribed from the expression cassette.

Utility

The above described methods of producing non-cellular nucleic acid libraries and the libraries produced thereby find use in a number of different applications. Representative applications of interest include, but are not limited to, functional genomic applications, in which the libraries are employed to determine the function of genes, e.g., in a high throughput manner. Such applications include those described in: U.S. Pat. Nos. 5,679,523 and 6,413,776; as well as published PCT Application Nos. WO 02/070684; WO 02/092807 and WO 02/092808, the disclosure of which patents and published applications, and/or corresponding United States priority documents and applications, are incorporated herein by reference.

One representative specific functional genomic application of interest in which the above methods and libraries find use is random homozygous gene inactivation, in which gene function is identified through random silencing of a gene and identification of a resultant phenotype of interest, which phenotype is then employed to assign functionality to the silenced gene.

The cellular library that is screened according to the subject methods may be produced using any convenient protocol, where representative protocols for preparing cellular libraries of antisense nucleic acids for use in functional genomic screening assays are reviewed in the specific patents and applications listed above. Such protocols may include the production of randomly integrating retroviral particular vectors, e.g., through placement of the library into an appropriate viral expression vector which is then introduced into a packaging cell for production of infective viral particles, etc.

The nature of the cell into which the library is placed may vary. In many embodiments, the cells into which the library to be screened is introduced are eukaryotic cells, such as plant cells, insect cells, fish cells, fungal cells, mammalian cells, and the like. Where the cells are mammalian cells, mammalian cells of interest include, but are not limited to: mouse cells, rat cells, primate cells, e.g., sequentially human cells, and the like.

The library may be introduced into the target cell population using any convenient protocol. For example, the constructs may be introduced by retroviral infection, electroporation, fusion, polybrene, lipofection, calcium phosphate precipitated DNA, or other conventional techniques. Particularly, the construct is introduced by viral infection for largely random integration of the construct in the genome. The construct is introduced into cells by any of the methods described above.

The cells of the resultant cellular library, e.g., produced as described above, are then assayed or screened for a cell phenotype of interest, e.g., a cell phenotype distinguishable from the wild-type phenotype. Different types of phenotypes may include changes in growth pattern and requirements, sensitivity or resistance to infectious agents or chemical substances, changes in the ability to differentiate or nature of the differentiation, changes in morphology, changes in response to changes in the environment, e.g., physical changes or chemical changes, changes in response to genetic modifications, and the like.

For example, the change in cell phenotype may be the change from normal cell growth to uncontrolled cell growth. The cells may be screened by any convenient assay which provides for detection of uncontrolled cell growth. One assay that may be used is a methylcellulose assay with bromodeoxyuridine (BrdU). Another assay that is effective is the use of growth in agar (0.3 to 0.5%>thickening agent). A test for tumorigenicity may also be used, where the cells may be introduced into a susceptible host, e.g., immunosuppressed, and the formation of tumors determined.

Alternatively, the change in cell phenotype may be the change from a normal metabolic state to an abnormal metabolic state. In this case, cells are assayed for their metabolite requirement, such as amino acids, sugars, cofactors, or the like, for growth. Initially, about 10 different metabolites may be screened at a time to assay for utilization of the different metabolites. Once a group of metabolites has been identified that allows for cell growth, where in the absence of such metabolites the cells do not grow, the metabolites are screened individually to identify which metabolite is assimilable or essential.

Alternatively, the altered cell phenotype may be a change from the ability of a cell to support the propagation of, or be subject to the pathogenic effects of, microorganisms such as viruses or bacteria to resistance to infection, propagation, or pathogenicity of these disease agents. Alternatively, the change may be from susceptibility to the injurious effects of toxins, such as anthrax or ricin to resistance to these effects.

Alternatively, the change in cell phenotype may be a change in the structure of the cell. In such a case, cells might be visually inspected under a light or electron microscope.

The change in cell phenotype may be a change in the differentiation program of a cell. For example, the differentiation of myoblasts to adult muscle fibers can be investigated. The differentiation of myoblasts can be induced by an appropriate change in the growth medium and can be monitored by determining the expression of specific polypeptides, such as myosin and troponin, which are expressed at high levels in adult muscle fibers.

The change in cell phenotype may be a change in the commitment of a cell to a specific differentiation program. For example, cells derived from the neural crest, if exposed to glucocorticoids, commit to becoming adrenal chromaffin cells. However, if the cells are exposed instead to fibroblast growth factor or nerve growth factor, the cells eventually become sympathetic adrenergic neuronal cells. If the adrenergic neuronal cells are further exposed to ciliary neurotrophic factor or to leukemia inhibitory factor, the cells become cholinergic neuronal cells. Cells transfected by the method of the subject invention can therefore be exposed to either glucocorticoids or any of the factors, and changes in the commitment of the cells to the different differentiation pathways can be monitored by assaying for the expression of polypeptides associated with the various cell types.

After identifying a cell in the library having a change in phenotype of interest and ascribing the change to the introduced nucleic acid library member therein, particularly to the region knocked out or silenced by antisense RNA encoded by the library member present in the cell, the silenced region may be characterized as desired, e.g., the region may be sequenced, the coding region may be used in the sense direction and a polypeptide sequence obtained. The resulting peptide may then be used for the production of antibodies to isolate the particular protein. Also, the peptide may be sequenced and the peptide sequence compared with known peptide sequences to determine any homologies with other known polypeptides. Various techniques may be used for identification of the gene at the locus and the protein expressed by the gene, since the subject methodology provides for a marker at the locus, obtaining a sequence which can be used as a probe and, in some instances, for expression of a protein fragment for production of antibodies. If desired the protein may be prepared and purified for further characterization.

The above described representative random homozygous gene inactivation applications find use in the identification of a genomic coding sequence of interest whose lack of expression resulting from the antisense mediated gene inactivation results in a phenotype of interest, as described above.

As such, the subject methods find use in a number of functional genomics applications, where specific applications in which such methods find use include, but are not limited to: gene target discovery applications, e.g., where identified gene targets may find use in the development of diagnostic products, therapeutic products, and the like.

ARAP3 Function and Methods of Modulating ARAP3 Expression/Activity

Exemplifying the power of the methods described above is the of the subject methods in the identification of the ARAP3 as having a function in Anthrax susceptibility. A nucleic acid encoding ARAP3, and the ARAP3 product encoded thereby, is deposited with GENBANK at accession no. AJ310567. The gene is obtained as a chromosomal fragment, where it is less than about 100 kbp, usually less than about 50 kbp, or as cDNA. The ARAP3 coding sequence will usually be flanked by nucleic acid sequences other than the sequences present at its natural chromosomal locus, where the different sequence will be within 10 kbp of the ARAP3 coding sequence. The protein may be obtained in purified form freed of other proteins and cellular debris, generally being at least about 50 weight % of total protein, more usually at least about 75 weight % of total protein, more usually at least about 95 weight % of total protein, and up to 100%. Similarly the nucleic acid encoding sequences, including fragments of at least 18 bp, more usually at least 30 bp, will be obtained in analogous purity, except that the percentages are based on total nucleic acids, comparing nucleic acid molecules having ARAP3 coding sequences to nucleic acid molecules lacking such sequences.

The inhibition of ARAP3 expression or activity results in an anthrax resistant phenotype. Therefore, the gene may be used in a variety of ways. The gene can be used for the expression and production of ARAP3 to identify agents which inhibit ARAP3 to determine the role that ARAP3 plays in the anthrax resistant phenotype. ARAP3 may be used to produce antibodies, antisera or monoclonal antibodies, for assaying for the presence of ARAP3 in cells. The DNA sequences may be used to determine the level of mRNA in cells to determine the level of transcription. In addition, the gene may be used to isolate the 5′ non-coding region to obtain the transcriptional regulatory sequences associated with ARAP3. By providing for an expression construct which includes a marker gene under the transcriptional control of the ARAP3 transcriptional initiation region, one can follow the circumstances under which ARAP3 is turned on and off.

Fragments of the ARAP3 gene may be used to identify other genes having homologous sequences using low stringency hybridization and the same and analogous genes from other species, such as primate, particularly human, and the like.

The ARAP3 gene or fragments thereof may be introduced into an expression cassette for expression or production of antisense sequences, where the expression cassette may include upstream and downstream in the direction of transcription, a transcriptional and translational initiation region, the ARAP3 gene, followed by the translational and transcriptional termination region, where the regions will be functional in the expression host cells. The transcriptional region may be native or foreign to the ARAP3 gene, depending on the purpose of the expression cassette and the expression host. The expression cassette may be part of a vector, which may include sites for integration into a genome, e.g., LTRs, homologous sequences to host genomic DNA, etc., an origin for extrachromosomal maintenance, or other functional sequences.

Therapeutic Applications of ARAP3 Expression/Activity Modulation

The methods find use in a variety of therapeutic applications in which it is desired to modulate, e.g., increase or decrease, ARAP3 expression/activity in a target cell or collection of cells, where the collection of cells may be a whole animal or portion thereof, e.g., tissue, organ, etc. As such, the target cell(s) may be a host animal or portion thereof, or may be a therapeutic cell (or cells) which is to be introduced into a multicellular organism, e.g., a cell employed in gene therapy. In such methods, an effective amount of an active agent that modulates ARAP3 expression and/or activity, e.g., enhances or decreases ARAP3 expression and/or activity as desired, is administered to the target cell or cells, e.g., by contacting the cells with the agent, by administering the agent to the animal, etc. By effective amount is meant a dosage sufficient to modulate ARAP3 expression in the target cell(s), as desired.

In the subject methods, the active agent(s) may be administered to the targeted cells using any convenient means capable of resulting in the desired modulation of ARAP3 expression and/or activity. Thus, the agent can be incorporated into a variety of formulations, e.g., pharmaceutically acceptable vehicles, for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. oligonucleotide decoy, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

The subject methods find use in the treatment of a variety of different conditions in which the modulation, e.g., enhancement or decrease, of ARAP3 expression and/or activity in the host is desired. By treatment is meant that at least an amelioration of the symptoms associated with the condition afflicting the host is achieved, where amelioration is used in a broad sense to refer to at least a reduction in the magnitude of a parameter, e.g. symptom (, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

In certain embodiments, the methods of ARAP3 modulation are methods of inhibiting ARAP3. Such methods find use in, among other applications, the treatment and/or prevention of anthrax related complications, and analogous disease conditions.

In these methods, modulation, e.g., inhibition of ARAP3 expression/activity may be accomplished using a number of different types of agents.

In certain embodiments, naturally occurring or synthetic small molecule compounds of interest include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below.

In yet other embodiments, expression of the ARAP3 is inhibited. Inhibition of ARAP3 expression may be accomplished using any convenient means, including use of an agent that inhibits ARAP3 expression (e.g., antisense agents, agents that interfere with transcription factor binding to a promoter sequence of the target ARAP3 gene, etc,), inactivation of the ARAP3 gene, e.g., through recombinant techniques, etc.

For example, antisense molecules can be used to down-regulate expression of the target protein in cells. The anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to the mRNA of the targeted protein, and inhibits expression of the targeted protein. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al, supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

In another embodiment, the ARAP3 protein gene is inactivated so that it no longer expresses a functional protein. By inactivated is meant that the gene, e.g., coding sequence and/or regulatory elements thereof, is genetically modified so that it no longer expresses functional repressor protein. The alteration or mutation may take a number of different forms, e.g., through deletion of one or more nucleotide residues in the region, through exchange of one or more nucleotide residues in the region, and the like. One means of making such alterations in the coding sequence is by homologous recombination. Methods for generating targeted gene modifications through homologous recombination are known in the art, including those described in: U.S. Pat. Nos. 6,074,853; 5,998,209; 5,998,144; 5,948,653; 5,925,544; 5,830,698; 5,780,296; 5,776,744; 5,721,367; 5,614,396; 5,612,205; the disclosures of which are herein incorporated by reference.

Also provided by the subject invention are screening assays designed to find modulatory agents of ARAP3 activity, e.g., inhibitors or enhancers of ARAP3 activity, as well as the agents identified thereby, where such agents may find use in a variety of applications, including as therapeutic agents, as described above. The screening methods may be assays which provide for qualitative/quantitative measurements of ARAP3 activity in the presence of a particular candidate therapeutic agent. The screening method may be an in vitro or in vivo format, where both formats are readily developed by those of skill in the art. Depending on the particular method, one or more of, usually one of, the components of the screening assay may be labeled, where by labeled is meant that the components comprise a detectable moiety, e.g. a fluorescent or radioactive tag, or a member of a signal producing system, e.g. biotin for binding to an enzyme-streptavidin conjugate in which the enzyme is capable of converting a substrate to a chromogenic product.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used.

A variety of different candidate agents may be screened by the above methods. As reviewed above, candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Using the above screening methods, a variety of different therapeutic agents may be identified. Such agents may target ARAP3 itself, or an expression regulator factor thereof. Such agents may be inhibitors or promoters of ARAP3 activity, where inhibitors are those agents that result in at least a reduction of ARAP3 activity as compared to a control and enhancers result in at least an increase in ARAP3 activity as compared to a control. Such agents may be find use in a variety of therapeutic applications, as reviewed above.

The following examples are offered by way of illustration and not by way of limitation.

### Experimental

The following experiments demonstrate the utilization of the antisense EST homozygous gene inactivation approach in identifying genes whose inactivation leads to cellular resistance to anthrax toxin. Preparation of vector constructs, methods for library production, assays for cellular resistance to anthrax toxin, and methods for isolating and analyzing the new gene are provided.

I. Materials & Methods:

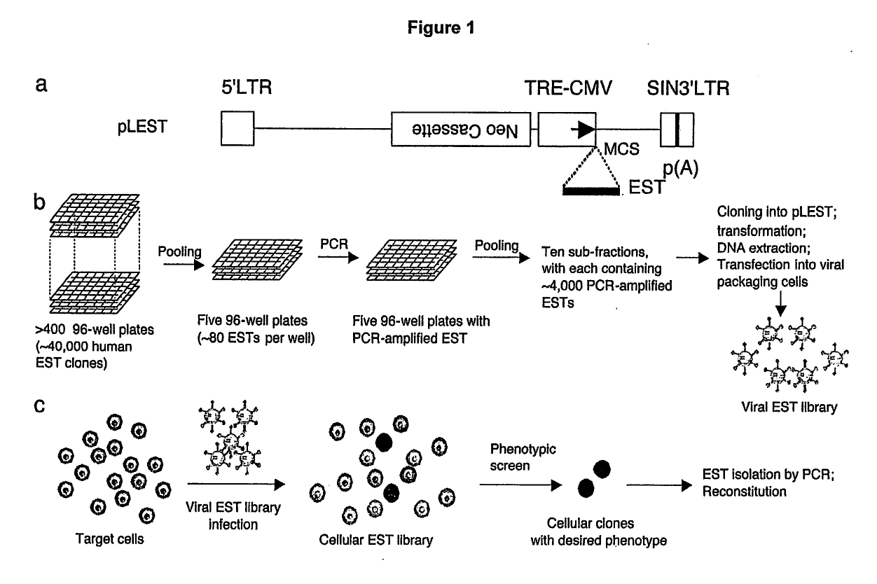

A. pLEST Vector. We constructed the EST expression vector pLEST by using a parental vector (pRRLsinPPT.CMV.MCS.Wpre, kindly provided by L. Naldini, University of Torino Medical School, Candiolo, Italy), which has been used for gene therapy (Follenzi et al., Nat. Genet. (2000) 25:217-222). We replaced the CMV promoter in the original vector backbone with a fused DNA fragment containing a neomycin-resistance expression cassette and a tetracycline-regulated tetracycline-responsive element (TRE)-CMV promoter. We obtained the neo cassette by SalI and BamHI digestion from the pCDNA-neo vector (Clontech) and the TRE-CMV promoter by XhoI and BamHI digestion from pRevTRE (Clontech). The neo cassette was placed in an orientation opposite to the direction of lentiviral gene transcription to prevent truncation of viral genomic RNA transcripts by the neo mRNA termination signal. A depiction of the pLEST vector is provided in FIG. 1a.

B. EST Library Construction. We obtained a human EST collection (Invitrogen) containing DNAs of ≈40,000 sequence-verified ESTs from the IMAGE Consortium. We removed ≈100 ng of DNA from each sample, pooled these DNAs into 96 subfractions that each contained 417 ESTs, and amplified the pooled EST DNA by PCR (18 cycles of 95° C. for 30 sec, 55° C. for 1 min, and 72° C. for 2 min; Hot-start, Qiagen, Valencia, Calif.). We used the following primers: ESTF_NheI, 5′-TCTGCTAGCCACACAGGAAACAGCTATG (SEQ ID NO:01); and ESTR_NheI, 5′-TCTGCTAGCTTGTAAAACGACGGCCAGTG (SEQ ID NO:02). The PCR products from the 96 sub-EST fractions were collected into 10 final groups, digested with NheI, and cloned by using the pLEST vector. We introduced the ligated DNA mixtures into XL2 blue Super-competent bacteria cells (Stratagene) and transferred the transformation mixtures into liquid LB medium containing ampicillin. This process is illustrated in FIG. 1B. A small fraction of the mixture was removed to estimate the size of the library (i.e., number of independent clones), and 3 ml of the culture was frozen as stock. The remaining portion of the culture was used for DNA preparation (Maxi DNA kit, Qiagen). Before carrying out the procedures described above, the ability of Escherichia coli libraries containing a collection of human ESTs to maintain the initial EST representation during library construction was estimated in a pilot experiment by using small subpool containing 100 ESTs. Sequencing of EST inserts from 20 randomly selected individual pLEST-containing bacterial clones after amplification of the subpool revealed one repeat sequence among this population.

C. Genomic DNA Extraction and PCR. We isolated genomic DNA from 1-2 million cultured cells of individual clones by using the Gentra genomic-DNA-extraction kit (Gentra Systems). Genomic DNA usually was dissolved in 50 μl of the DNA-hydration buffer. PCR-amplification of the EST insert used the following primers: ESTF, 5′-CACACAGGAAACAGCTATG (SEQ ID NO:03); and ESTR, 5′-TTGTAAAACGACGGCCAGTG (SEQ ID NO:04). Gel-purified PCR products were sequenced by using either one of the EST primers. To determine the orientation of the inserted EST, we performed genomic PCR by using one of the EST primers and Lenti3 primer (5′-TGTTGCTCCTTTTACGCTATG) (SEQ ID NO:05), which is located 3′ of the EST insert in the pLEST vector.

D. Mammalian Cell Culture and Transfection. We maintained the prostate cancer cell line M2182 (kindly provided by J. L. Ware, Medical College of Virginia, Richmond) in RPMI 1640 medium (Invitrogen) by using supplements as described in Jackson-Cook et al., Cancer Genet. Cytogenet. (1996) 87:14-23. We cultured the Raw 264.7 mouse macrophage and 293T cell lines in DMEM (Invitrogen) containing 10% FBS. We performed DNA transfections with Lipofectamine 2000 (Invitrogen) or FuGene6 (Roche) according to the manufacturer's recommended protocols.

E. Lentivirus Production and Infection. We produced lentivirus by transient transfection of 293T cells (calcium phosphate precipitation method) by using library DNA along with DNAs of packaging and VSVG envelope constructs as described (Follenzi et al., supra). Cells were supplied with fresh medium 24 h after transfection, and virus-containing supernatant collected 24 h after this medium change was filtered through a 0.22-μm low-protein binding filter (Millipore). Infection of cells by the filtered lentivirus was carried out in suspensions containing polybrene at 37° C. for 6-18 h; selection for virus-infected cells was carried out by adding the antibiotic G418 (Invitrogen) 48 h after the start of infection (the G418 dosage was 350 μg/ml for M2182 cells and 500 μg/ml for Raw 264.7 cells). G418-resistant clones were pooled 10-14 d later. Library size was estimated by counting the number of independent G418-resistant clones on each plate before the pooling.

F. Western Blotting. Rabbit polyclonal anti-PA antibody (1:1,000 dilution) and goat polyclonal anti-ARAP3 antibodies (1:1,000 dilution) were kindly provided by S. Leppla (National Institute of Allergy and Infectious Diseases, National Institutes of Health, Bethesda) and P. Hawkins (The Babraham Institute, Cambridge, United Kingdom), respectively. Mouse anti-tubulin mAb and horseradish peroxidase-conjugated secondary antibodies were purchased from Santa Cruz Biotechnology. Western blotting was performed essentially as described (Harlow & Lane, Using Antibodies: A Laboratory Manual (Cold Spring Harbor Press, 1999)). Chemiluminescence of Western blot bands was quantitated by using a Versadoc 1000 instrument (Bio-Rad).

G. Toxin Treatment. PA and LF were purchased from List Biological Laboratories (Campbell, Calif.). FR59 was a gift from S. Leppla. We exposed cells to toxins for 48 h unless otherwise indicated; we used 50 ng/ml PA plus 50 ng/ml FP59 to treat M2182 cells. We used 500 ng/ml PA plus 500 ng/ml LF to constitute the native anthrax toxin in experiments employing Raw 264.7 cells. After toxin treatment, cells were washed with PBS and cultured in fresh growth medium for up to 2 weeks to identify surviving clones or for 1 d before testing in 3-(4,5-dimethylthiazol-2-yl)-2,5-diphenyl tetrazolium bromide (MTT) assays.

H. MTT Viability Assay. Cells were seeded and treated the next day with the indicated amount of toxin. After incubation at 37° C. (2 d for M2182 cells, and 3 h for Raw 264.7 cells), cells were washed, supplied with fresh medium, and cultured for additional 24 h. We then added 10 μl of MTT (Sigma) freshly prepared at 10 mg/ml in PBS to cells, incubated the cells at 37° C. for 2 h, removed the supernatant, added 50 μl of lysis buffer (10% SDS/0.01 M HCl), and continued incubation at 37° C. for 10 min. We added 200 μl of PBS to cell lysates, and absorbance readings at 570 nm (Tecan Technologies, Research Triangle Park, N.C.) were obtained immediately.

I. Assays for Processing of PA. We performed these assays according to a protocol in Liu & Leppla, J. Biol. Chem. (2003) 278:5227-5234. To assess binding of PA to the cell surface, cells plated 1 d previously at 70-80% confluence were cooled to room temperature for 15 min, washed with PBS once, and incubated with 1 μg/ml PA in binding buffer (DMEM without carbonate/25 mM Hepes/50 μg/ml gentamycin/0.5 mg/ml BSA, pH 7.4) at 4° C. for 2 h. We then washed the cells with cold PBS four to five times and disrupted them in lysis buffer (150 mM NaCl/50 mM Tris.HCl, pH 7.5/0.5% Nonidet P-40). Lysates were examined by Western blotting using anti-PA antibody and anti-tubulin antibody as probes. For PA internalization, we treated cells at 70-80% confluence with 1 μg/ml PA at 37° C. for 30 min, rinsed the cells with cold PBS once, and trypsinized and washed the cells with PBS three times. Cellular lysates were made and examined by Western blotting as indicated above.

J. Fluorescence Confocal Microscopy. PA protein was first labeled with Alexa Fluor 488 by using the A-10235 protein-labeling kit (Molecular Probes). The potency of PA after labeling proved to be retained, as determined by MTT assay. Immunostaining was performed as follows. In brief, cells were grown on cover slips, incubated with or without 0.5 μg/ml PA-Alexa 488 for 30 min at 4° C. for PA-binding analysis and at 37° C. for PA-processing analysis, washed with PBS for three times, fixed in 4% paraformaldehyde, and permeabilized by addition of 0.2% Triton X-100. The cells were then mounted onto slides and examined by using an LSM confocal microscope (Zeiss).

II. Results

A. Construction of Lentivirus-Based Antisense EST Libraries. To enable efficient and controllable expression of ESTs, we constructed the lentivirus-based expression vector pLEST. A schematic diagram of the vector is shown in FIG. 1a. The backbone of pLEST is derived from lentiviral vector RLsinPPT.CMV.MCS.Wpre (Follenzi et al., supra) but lacks its constitutive promoter. Instead, pLEST contains a TRE-regulated CMV promoter, allowing tetracycline-regulated gene transcription of ESTs introduced into the vector. pLEST also carries a neomycin (neo)-resistance cassette, which confers G418 drug resistance in mammalian cells and, thus, can act as a selectable marker for stable integration of pLEST into the chromosomes of vector-infected cells. The neo cassette is placed in an orientation opposite to the direction of transcription of the RNA that comprises the lentiviral genome so that the viral genome transcript will not be terminated prematurely. In addition, pLEST contains a multiple cloning site (MCS) for insertion of ESTs.

By using pLEST, we constructed a library of lentiviruses that express ≈40,000 previously cloned ESTs representing ≈28,000 unique human genes (FIG. 1b). Because the EST sequences were inserted bidirectionally in the expression vector, we anticipated that the lentivirus-based EST library would be capable of inactivating complementary mRNAs by antisense mechanisms and, possibly, also of interfering with the functions of some proteins by the production of dominant-negative peptide fragments encoded by ESTs transcribed in the sense direction.

B. Isolation of Cellular Clones Resistant to PA-Dependent Toxicity. The genetic screen that we used was designed to identify human cell clones that show reduced toxin sensitivity after infection with the lentivirus-based EST library described above (FIG. 1c). A human prostate cancer cell line M2182 (Jackson-Cook et al., supra) that was engineered to express the tetracycline-dependent transcriptional activator (tTA) was infected with this library, yielding approximately 1 million independent G418-resistant clones. Our initial screenings used a hybrid toxin consisting of PA and a recombinant cytotoxin FP59; FP59 is a fusion protein containing the N-terminal PA-binding domain of LF and the ADP-ribosylation domain of Pseudomonas aeruginosa exotoxin A (Arora et al., J. Biol. Chem. (1992) 267:15542-15548)). Because the lethality of FP59 requires PA-mediated cellular entry of the exotoxin component, we anticipated that survivors would include clones in which this function of PA is defective. After exposure of the M2182 cellular EST library to PA plus FP59, 20 surviving cell colonies were observed, whereas fewer than five survivors were present in similarly sized control populations infected with a lentivirus vector lacking EST inserts. Retesting of survivors from the EST-expressing population indicated maintenance of toxin resistance in 15 of the 20 EST-infected isolates.

Three of the PA/FP59-resistant clones, including one that we designated as F7, showed decreased resistance in the presence of doxycycline, which is a tetracycline analog that down-regulates the TRE-CMV promoter. Reversal of the resistance phenotype was incomplete, possibly because repression of the promoter is only partial (cf., Zhu et al., J. Biol. Chem. (2001) 276:25222-25229)). To determine the specificity of the toxin-resistance phenotype in these clones, we performed MTT cell-viability assays by using serially diluted toxins. Although all three tetracycline-reversible clones were resistant to the PA/FP59 hybrid toxin, only clone (F7) exhibited a phenotype specific for PA/FP59; the clone F7 cells were as sensitive as naïve wild-type cells to native Pseudomonas exotoxin and diphtheria toxin, neither of which depends on PA for cellular entry. These results showed that the decreased PA/FP59 toxin sensitivity observed for clone F7 results from interference with functions specifically mediated by the PA component of the hybrid toxin.

C. Clone F7 Expresses Antisense ARAP3 EST and Contains Reduced ARAP3 Protein. The EST expressed in clone F7 was amplified by PCR from genomic DNA using two primers complementary to vector sequences bracketing EST inserts. Sequence analysis of the single PCR product that we obtained indicated that it corresponds to a segment (nucleotides 998-1,458; IMAGE clone no. 809620) of cDNA encoding ARAP3, a recently described phosphoinositide-binding protein that includes a GTPase-activating protein (GAP) domain for Arf6-GTPase and another GAP domain for Rho GTPase (Krugman et al., Mol. Cell. (2002) 9:95-108; Santy & Casanova, Curr. Biol. (2002) 12:R360-R362)). ARAP3 has been shown to be a specific phosphoinositide-stimulated Arf6 GAP, and both of its GAP domains play a role in mediating PI3K-dependent rearrangements in the cell cytoskeleton and cell shape (Krugman et al., supra). Further analysis of the PCR product amplified from F7 indicated that the ARAP3 EST in this toxin-resistant cell clone was oriented in antisense direction relative to the TRE-CMV promoter. Western blotting quantitated by chemiluminescence densitometry showed that ARAP3 protein expression in F7 was reduced to ≈30% of the level observed in the parental M2182 cell line. Transient overexpression of an ARAP3 protein fused at the N terminus with GFP partially reversed the increased resistance of F7 cells to PA/FP59.

D. Expression of Antisense ARAP3 EST in Naïve Cells Recapitulates Toxin-Resistance Phenotype. The role of ARAP3 deficiency in toxin resistance was confirmed by experiments in which the ARAP3 EST was cloned in antisense orientation in the pLEST vector and introduced into naïve M2182-tTA cells. Whereas control cells infected with the vector virus alone or expressing the ARAP3 EST in the sense direction were killed efficiently by PA/FP59, M2182 cells transcribing this EST in the antisense direction had reduced toxin susceptibility. Three randomly picked toxin-resistant clones that were isolated from this reconstitution experiment showed 70% reduction in ARAP3 protein. We were unable to reduce the ARAP3 protein to a level that was comparable with that in F7 cells by using stable small interfering RNA in naïve M2182 cells. Although we obtained partial reversion of toxin resistance in clone F7 by transient overexpression of ARAP3, we were unable to establish stable F7-derived cell lines that expressed ARAP3 to a level that was sufficient to overcome the effects of antisense-mediated inhibition of ARAP3 expression fully.

In vivo, macrophages are one of the targets of anthrax infection, and in culture, they are susceptible to killing by anthrax lethal toxin (LeTx) formed by the interaction of PA with LF (Weinrauch & Zychlinsky, Annu. Rev. Microbiol. (1999) 53:155-187; Dixon et al., Cell Microbiol. (2000) 2:453-463)). Paralleling its role in the internalization of FP59 in the experiments described above, PA mediates entry of LF into macrophages. We introduced the tTA element into the mouse macrophage cell line Raw 264.7, infected these cells with a lentivirus expressing the ARAP3 human EST in antisense orientation, and investigated both macrophage susceptibility to LeTx and the effect of these manipulations on the cellular level of ARAP3 protein. Antisense expression of the human EST sequence, which has 92% identity to the corresponding segment of mouse ARAP3 transcript resulted in ≈60% reduction of ARAP3 protein and ≈2-fold enhancement of cellular resistance to LeTx treatment as determined by MTT assay.

E. ARAP3-Deficient Cells Exhibit Impaired PA Internalization. Collectively, the above results show that the toxin-resistance phenotype produced by ARAP3 deficiency results from impaired functioning of PA, which in our experiments was required as a carrier by both FP59 and LF. To evaluate this interpretation further and to understand the mechanism(s) underlying our findings, we investigated the effects of ARAP3 deficiency on certain parameters of PA function (membrane binding, cleavage, and internalization of PA oligomers). We observed no detectable alteration of PA membrane binding in either clone F7 or reconstituted M2182 cells expressing antisense RNA to the ARAP3 EST, as indicated by the intensity of the unprocessed 83-kDa PA band. However, in ARAP3-deficient cells that were incubated with PA at 37° C., which ordinarily enables internalization of oligomers of the cleaved 63-kDa PA subunit (Liu et al., supra), the intracellular level of PA oligomers was reduced to approximately one-third of normal, as determined by densitometry analysis of the Western blot. Defective internalization of PA in F7 cells was confirmed by fluorescence microscopy using FITC-labeled PA, whereas nearly all of the PA-associated fluorescence signal entered the cytoplasm in naïve cells and was detectable in the form of cytosolic aggregates after a 30-min period of incubation at 37° C., consistent with the results given in Abrami et al., J. Cell. Biol. (2003) 160:321-328, more than one-half of the PA signal remained on the cell surface in cells of clone F7.

III. Discussion

A. The above results demonstrate the utility of the EST-based approach of the subject invention for global inactivation of host genes, where the subject methodology is useful as a general loss-of-function genetic screen. The above results also show that overall, the equal representation of ESTs employed as the starting material in the methods of the subject invention is maintained during the pooling, PCR-amplification, cloning and transformation process steps of the subject methods. This discovery that various clones in the EST library maintain this original representation and that EST libraries thus do not become unbalanced by possible selective growth of some clones is highly important to the utility of this invention.

Advantages of the EST-based gene-inactivation approach described here are the predefined composition of and approximately equal representation of genes in EST libraries, in addition to the opportunity for genome-wide coverage by a single library prepared from already available ESTs corresponding to variably spliced transcripts from multiple tissues. ESTs producing a phenotype of interest can be identified rapidly by using one-step PCR amplification of genomic DNA from the functionally altered cells. Microarray analysis of gene expression in several independent clones of cells targeted by our antisense EST libraries showed no detectable evidence of induction of IFN-response genes (Q.L. and S.N.C., unpublished data).

Accordingly, with respect to gene inactivation assays, the subject invention provides a number of advantages over other methods of gene inactivation. One major advantage the subject EST approach is its equal representation of genes in the library. This feature allows maximal gene coverage for a library with a reasonable size, and thus increases the efficiency of the library construction and screening process. A second advantage of the subject system arises from the feature that ESTs are collected from all sorts of different tissues and consequently reflect genome-wide expression. Therefore, a single broad-based EST library can be made and investigated in many different cell types. In contrast, the conventional cDNA approach commonly involves a series of cDNA libraries that utilize mRNAs isolated from multiple types of cells in an effort to achieve genome-wide coverage. A third advantage of the subject system is its expandability and flexibility. For example, newly identified ESTs can be easily added to the existing library. Accordingly, the present invention represents a significant contribution to the art.

B. The above results and discussion also demonstrate that ARAP3 has a function in Anthrax susceptibility, such that Anthrax susceptibility can be modulated through modulation of ARAP3 expression. The above results and discussion demstrate a role for ARAP3 in the processing (particularly the internalization) of the anthrax protective antigen. The above results and discussion also demonstrate that inhibition of ARAP3 expression results in an anthrax resistant phenotype. As such, inhibition of ARAP3 results in anthrax resistance, and is a way to prevent and/or treat complications arising from anthrax exposure.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A predefined pooled collection of distinct nucleic acid vectors, wherein each constituent member of said pooled collection comprises an expression cassette that corresponds to a chromosomal transcript of known sequence.

2. The pooled collection according to claim 1, wherein said collection comprises at least 100 distinct nucleic acid vectors.

3. The pooled collection according to claim 2, wherein said collection comprises at least 1000 distinct nucleic acid vectors.

4. The pooled collection according to claim 1, wherein said pooled collection is a library of ESTs.

5. A method of reducing expression of one or more chromosomal coding regions in a population of cells, said method comprising contacting said population of cells with a pooled collection of vectors according to claim 1.

6. The method according to claim 5, wherein said method is a method of identifying a genomic coding sequence of interest.

7. The method according to claim 5, wherein said method is a method of determining function of a genomic coding sequence.

8. The method according to claim 5, wherein said collection comprises at least 100 distinct nucleic acid vectors.

9. The method according to claim 8, wherein said collection comprises at least 1000 distinct nucleic acid vectors.

10. The method according to claim 5, wherein said pooled collection is a library of ESTs.

11. A method of identifying a genomic coding sequence of interest, said method comprising:
(a) producing a non-cellular nucleic acid library by:
(i) dividing an initial set of a plurality of separate nucleic acids into two or more pooled collections having an initial sequence representation profile, wherein each pooled collection includes not more than about 100 distinct nucleic acids;
(ii) amplifying each of said pooled collections to produce two or more amplified pooled collections; and
(iii) combining said two or more amplified pooled collections to produce said non-cellular nucleic acid library, wherein said non-cellular nucleic acid library has a nucleic acid sequence representation profile that is substantially the same as said initial sequence representation profile;
(b) transforming a population of cells with said nucleic acid library to produce a cellular library; and
(c) identifying members of said cellular library that display a phenotype of interest to identify said genomic coding sequence of interest.

12. The method according to claim 11, wherein said non-cellular nucleic acid library is an EST library.

13. The method according to claim 11, wherein said non-cellular nucleic acid library is a library containing sequences complementary to at least a segment of a chromosomal transcript of a chromosomal transcript.

14. The method according to claim 13, wherein said phenotype of interest results from loss of function of said genomic coding sequence of interest.

15. The method according to claim 11, wherein said non-cellular nucleic acid library is a sense library.

16. The method according to claim 11, wherein said non-cellular nucleic acid library is present in a vector system.

17. The method according to claim 16, wherein said vector system is an integrating vector system.

18. The method according to claim 11, wherein said non-cellular nucleic acid library comprises a substantially equal amount of each constituent nucleic acid member.

19. The method according to claim 11, wherein said non-cellular nucleic acid library has a ratio of number of distinct nucleic acids to total amount of nucleic acid that ranges from about 10/μg to about 10,000/μg.

20. The method according to claim 19, wherein said non-cellular nucleic acid library comprises at least about 1000 distinct nucleic acids of different sequence.

21. A method of identifying a genomic coding sequence of interest, said method comprising:
(a) producing a non-cellular expressed sequence tag (EST) library by:
(i) dividing an initial set of a plurality of separate ESTs into two or more pooled collections of ESTs having an initial EST representation profile, wherein each pooled collection includes not more than about 100 distinct ESTs;
(ii) amplifying each of said pooled collections to produce two or more amplified pooled collections; and
(iii) combining said two or more amplified pooled collections to produce said non-cellular EST library, wherein said non-cellular EST library has an EST representation profile that is substantially the same as said initial EST representation profile;
(b) transforming a population of cells with said non-cellular EST library to produce an EST cellular library; and
(c) identifying cellular members of said cellular library that display a phenotype of interest to identify said genomic coding sequence of interest, wherein said phenotype of interest results from loss of function of said genomic coding sequence of interest.

22. The method according to claim 21, wherein said non-cellular EST library is present in a vector system.

23. The method according to claim 22, wherein said vector system is an integrating vector system.

24. The method according to claim 21, wherein said non-cellular EST library comprises a substantially equal amount of each constituent EST member.

25. The method according to claim 21, wherein said non-cellular EST library has a ratio of number of distinct ESTs to total amount of nucleic acid that ranges from about 10/μg to about 10,000/μg.

26. The method according to claim 25, wherein said non-cellular EST library comprises at least about 1000 distinct ESTs of different sequence.

27. A method of producing a non-cellular nucleic acid library, said method comprising:
(a) dividing an initial set of a plurality of separate nucleic acids into two or more pooled collections of nucleic acids having an initial sequence representation profile, wherein each pooled collection includes not more than about 100 distinct nucleic acids;
(b) amplifying each of said pooled collections to produce two or more amplified pooled collections; and
(c) combining said two or more amplified pooled collections to produce said non-cellular nucleic acid library, wherein said non-cellular nucleic acid library has a sequence representation profile that is substantially the same as said initial sequence representation profile.

28. The method according to claim 27, wherein said non-cellular nucleic acid library is an EST library.

29. The method according to claim 27, wherein said non-cellular nucleic acid library is a library containing sequences complementary to at least a segment of a chromosomal transcript of a chromosomal transcript.

30. The method according to claim 27, wherein said non-cellular nucleic acid library is present in a vector system.

31. The method according to claim 27, wherein said vector system is an integrating vector system.

32. The method according to claim 27, wherein said non-cellular nucleic acid library comprises a substantially equal amount of each constituent nucleic acid member.

33. The method according to claim 27, wherein said non-cellular nucleic acid library has a ratio of number of different nucleic acids to total amount of nucleic acid that ranges from about 10/μg to about 10,000/1μg.

34. The method according to claim 33, wherein said non-cellular nucleic acid library comprises at least about 1000 nucleic acids of different sequence.

35. A non-cellular nucleic acid library produced according to the method of claim 27.

36. A cellular nucleic acid library produced by transforming a population of cells with a non-cellular library according to claim 35.

37. A method of identifying a genomic coding sequence of interest, said method comprising:
(a) transforming a population of cells with a predefined pooled collection of distinct nucleic acid vectors, wherein each constituent member of said pooled collection comprises an expression cassette that corresponds to a chromosomal transcript of known sequence; and
(b) identifying members of said cellular library that display a phenotype of interest to identify said genomic coding sequence of interest.

38. A cellular member of said cellular library produced according to the method of claim 37.

39. The cellular member according to claim 38, wherein said cellular member displays a phenotype of interest that caused by inactivation of a genomic coding sequence by a nucleic acid vector member of said pooled collection.

40. A genomic coding sequence present in other than its natural environment identified according to the method of claim 37.

41. A nucleic acid transcript of a genomic coding sequence according to claim 40.

42. An expression product of a genomic coding sequence according to claim 40.

43. A method of treating a subject suffering from an anthrax mediated disease condition, said method comprising:
administering to said subject an effective amount of ARAP3 inhibitory agent to treat said subject.

44. A method of conferring an anthrax resistant phenotype on a subject, said method comprising:
administering to said subject an effective amount of an ARAP3 inhibitory agent.


SEQUENCE LISTING (5 sequences)

SEQ ID NO: 1 - DNA, 27 bp, human
ctgctagccacacaggaaacagctatg

SEQ ID NO: 2 - DNA, 29 bp, human
tctgctagcttgtaaaacgacggccagtg

SEQ ID NO: 3 - DNA, 19 bp, human
cacacaggaaacagctatg

SEQ ID NO: 4 - DNA, 20 bp, human
ttgtaaaacgacggccagtg

SEQ ID NO: 5 - DNA, 21 bp, lentivirus
tgttgctccttttacgctatg